(19) United States
(12) Patent Application Publication
(10) Pub. No.: US 2006/0228786 A1
(43) Pub. Date: Oct. 12, 2006

(54) Polymerase-based protocols for the introduction of deletions and insertions

(76) Inventor: John C Salerno, Averill Park, NY (US)

Correspondence Address: ELMORE PATENT LAW GROUP, PC, 209 MAIN STREET, N. CHELMSFORD, MA, 01863, US

(21) Appl. No.: 10/544,429

(22) PCT Filed: Feb. 6, 2004
(86) PCT No.: PCT/US04/03616

§ 371 Date: Dec. 27, 2005

Related U.S. Application Data
(60) Provisional application No. 60/445,689, filed on Feb. 6, 2003.
(60) Provisional application No. 60/445,703, filed on Feb. 6, 2003.
(60) Provisional application No. 60/446,045, filed on Feb. 6, 2003.
(60) Provisional application No. 60/445,704, filed on Feb. 6, 2003.
(60) Provisional application No. 60/474,063, filed on May 29, 2003.

(51) Int. Cl. C12P 19/34

(52) U.S. Cl. 435/91.2

(57) ABSTRACT

The present invention relates to improved methods of introducing site-directed mutations into circular DNA without the need for subcloning. The invention comprises single-stage, polymerase-based, mutagenesis procedures which eliminate the need for the second-stage linear cyclic amplification reaction of the prior art. In accordance with the invention, production of complimentary mutagenized DNA strands is accomplished in separate, single-primer, linear amplification reactions carried out for each primer of a mutagenic primer pair. The mutagenized DNA strands produced in the separate primer reactions are combined and annealed and the resulting double-stranded mutagenized DNA intermediate may then be used directly to transform a host cell for further production of the desired mutant DNA. Major applications of this method include directed evolution and other areas that benefit from the development of diversity.

### RELATED APPLICATION(S)

This application claims the benefit of U.S. Provisional Application No. 60/445,689, filed on Feb. 6, 2003, U.S. Provisional Application No. 60/445,703, filed on Feb. 6, 2003, U.S. Provisional Application No. 60/446,045, filed on Feb. 6, 2003, U.S. Provisional Application No. 60/445,704, filed on Feb. 6, 2003, and U.S. Provisional Application No. 60/474,063, filed on May 29, 2003, Docket No. RPI-812, entitled “Parental Suppression via Polymerase-based Protocols for the Introduction of Deletions and Insertions.” The entire teachings of the above applications are incorporated herein by reference.

### BACKGROUND OF THE INVENTION

In recent years a number of methods have come into common use that allow the generation of site directed mutants without subcloning based on polymerase activity. This technology is mature enough to allow the sale of a number of mutagenesis kits that are capable of producing point mutants and in some case insertion and deletion mutants (‘indels’).

One such mutagenesis system is supplied by Stratagene (La Jolla, Calif.) and is sold under the name QuikChange® Site Directed Mutagenesis Kit (QCM). The Strategene system is widely used and effective for the production of single codon mutations. In the Stratagene protocol shown in FIG. 1, complementary primers carrying the desired mutation produce linear copies of the original circular plasmids in a linear cyclic amplification reaction; these form duplexes with complementary overhanging ends which can be taken up by ultracompetent cells and nick repaired in vivo to produce functioning circular plasmids. The procedure also includes a dam methylation strategy to destroy parental strands to increase the frequency of mutation in the transformed colonies.

Wang and Malcolm (Methods Mol. Biol. (2002) 182:37-43) introduced an important modification to the Stratagene QCM method which greatly extended its capabilities. Wang and Malcolm described a two stage procedure for the production of mutants (see, FIG. 2). Their critical insight was the recognition that major mutations such as insertions and deletions caused the primers used in the QCM procedure to anneal to each other much better than they did to the parental gene. The production of primer dimers was therefore, almost complete, producing very low concentrations of mutant DNA using the QCM procedure alone. Wang and Malcolm attempted to solve this problem by the introduction of a single primer extension stage prior to a second stage linear cyclic amplification reaction (as defined below) in which each primer separately produces linear strands in an amplification reaction. After a few cycles of production of single-stranded mutant DNA, the products for the two primers are mixed and a second stage of linear cyclic amplification is carried out using the QCM procedure in the normal manner. The rational for adding the single primer extension stage was that the primers were now exact matches for the regions at the ends of the linear strands so that annealing could compete effectively with primer dimer formation. This method was shown to efficiently produce insertion and deletion mutants which could not be generated using the QCM procedure.

However, there are still common disadvantages to the Wang and Malcolm and the QCM procedures. In the Wang and Malcolm studies, using more than one or two cycles in the single primer extension stage produced fewer mutant colonies. The reason for this is that no productive reactions are possible in the second stage, but destructive reactions are possible and are made more likely as the first round is extended beyond 1-2 cycles. To understand this, it is necessary to appreciate that neither the QCM procedure or the Wang and Malcolm procedure ever carry out true PCR. The primers which are extended on the original circular plasmid framework produce linear copies which are not themselves templates for further DNA synthesis with those primers; hence, the amplification is linear, and is not the exponential amplification of a chain reaction which produces its own templates (true PCR). In fact, if the full length single strands resulting from the first stage primer extension anneal during the second stage linear cyclic amplification reaction, each strand can be extended by a polymerase at its 3′ end. This destroys the strands as transformation units because the gene is disrupted by a second copy of the primer, and because there are no sticky ends to allow the plasmid to be recircularized. This destructive extension is prevented by binding of the primers to their complements, however it is likely that the first stage primer extension produced an excess of double strands, destructive extension will predominate. Therefore, the second stage of linear amplification (essentially the QCM procedure) in accordance with the Wang and Malcolm protocol, is unproductive.

In addition, the extended strands produced in this reaction are perfect templates for a runaway PCR artifact which produces high concentrations of linear copies which are incapable of transformation, since each end of both strands carries a primer copy. These copies are indistinguishable on agarose gels from the active transforming species present at much lower concentration and are typically the dominant product.

Thus, a need still exists to further improve the efficiency of these methods.

### SUMMARY OF THE INVENTION

The present invention relates to improved methods of introducing site-directed mutations into circular DNA without the need for subcloning. The invention comprises single-stage, polymerase-based, mutagenesis procedures which eliminate the need for the second-stage linear cyclic amplification reaction of the prior art. In accordance with the invention, the elimination of the prior art second stage linear cyclic amplification reaction provides improved performance and efficiency over the prior art and allows extensive modifications of DNA, including insertions, deletions, point mutations and multiple point mutations. The elimination of the second stage linear cyclic amplification reaction of the prior art further simplifies the procedure and reduces the amount of primer and other reagent that would be necessary in a two stage process. In accordance with the invention, production of complimentary mutagenized DNA strands is accomplished in separate, single-primer, linear amplification reactions carried out for each primer of a mutagenic primer pair. The mutagenized DNA strands produced in the separate primer reactions are combined and annealed and the resulting double-stranded mutagenized DNA intermediate may then be used directly to transform a host cell for further production of the desired mutant DNA. The invention also provides kits for site-directed mutagenesis with high efficiency. The kits of the invention contain reagents and instructions required for carrying out the methods of the invention.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic of the Stratagene Quik Change Mutagenesis package with linear amplification of plasmid DNA during temperature cycling produces excess linear mutant strands.

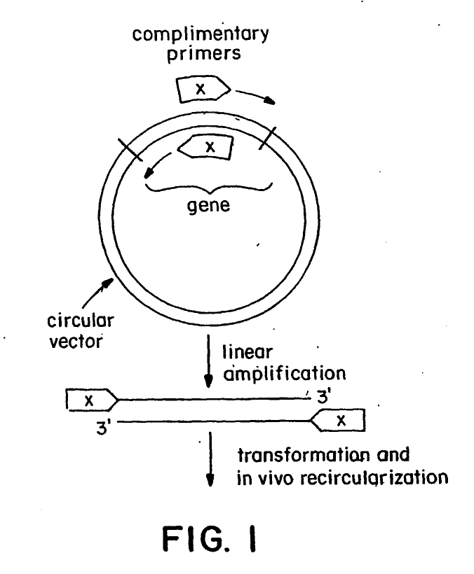

FIG. 2 is a schematic of Wang and Malcolm's modification of the Stratagene protocol.

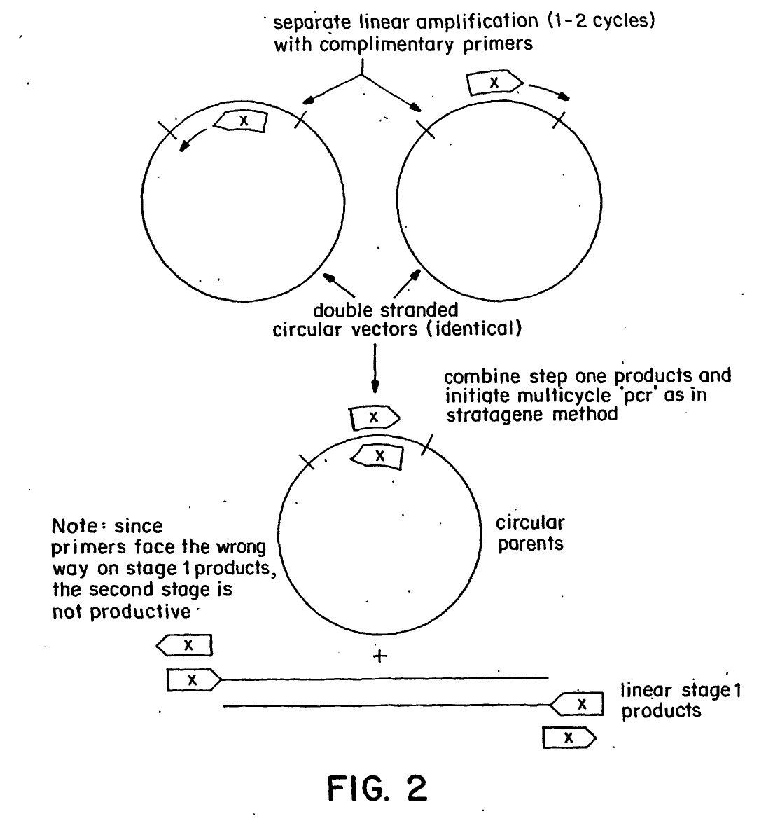

FIG. 3 is a schematic of the single-stage linear amplification protocol of the invention using separate samples for forward and reverse primers.

### DETAILED DESCRIPTION OF THE INVENTION

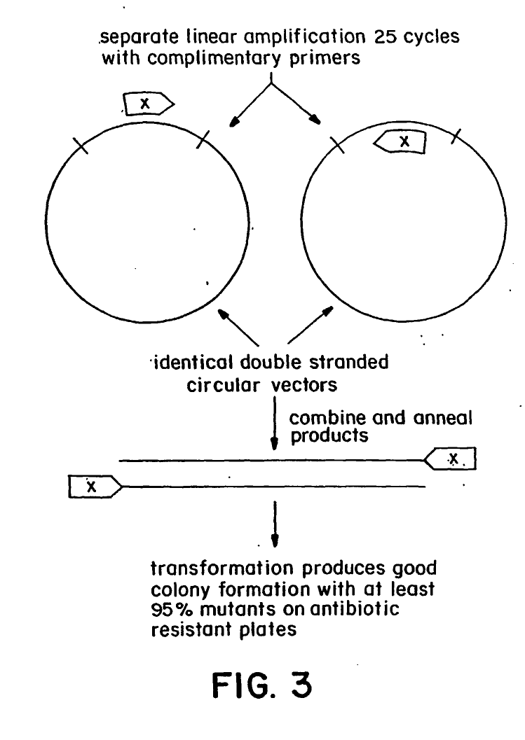

The present invention provides novel, improved methods for the creation of insertions, deletions, and point mutations and multiple point mutations in a DNA sequence of interest without subcloning and without the need for a second stage linear cyclic amplification reaction. A shown in FIG. 3, the method of the invention comprises the steps of:
(a) adding a first mutagenic primer to a first parental DNA molecule in a first reaction;
(b) adding a second mutagenic primer to a second parental DNA molecule in a second, reaction, wherein the first and second parental DNA molecules are identical, and wherein the first mutagenic primer comprises a region that is complementary to the second mutagenic primer and the first and second mutagenic primers each contain at least one mutation site with respect to the first and second parental DNA molecules, wherein the mutation site is located within the region that is complementary between said first and second mutagenic primers;
(c) synthesizing by means of at least one cycle of a single primer linear amplification reaction a first mutagenized DNA strand comprising the first mutagenic primer in the first reaction;
(d) synthesizing by means of at least one cycle of a single primer linear amplification reaction a second mutagenized DNA strand comprising the second mutagenic primer in the second reaction;
(e) combining the reaction products from (c) with the reaction products from (d);
(f) annealing the first mutagenized strand with the second mutagenized strand to form a double stranded mutagenized DNA intermediate.
   The resulting double-stranded mutagenized DNA intermediate may be used to transform competent or ultracompetent cells for nick repair and expression of the mutation.

In one embodiment of the invention, the method optionally comprises reacting a ligase with the double stranded mutagenized DNA intermediate for in vitro nick repair and recircularization prior to transformation of the host cell. In another embodiment, the invention optionally comprises the use of selection enzymes to digest the first and second parental DNAs after the synthesis steps or after the annealing step.

The terms “first parental DNA” and “second parental DNA” as used herein refer to DNA vectors comprising a vector region having a cloning site and further comprising a target sequence for mutagensis such as a gene, gene region, or another DNA of interest, inserted within the cloning site. In accordance with the present invention, the first and second parental DNAs are identical in that they have identical vectors, identical cloning sites, and an identical target sequence present within the cloning site.

The terms “linear amplification reaction,” and “single-primer linear amplification reaction” as used herein, refer to a variety of enzyme mediated polynucleotide synthesis reactions that employ pairs of polynucleotide primers to linearly amplify a given polynucleotide and proceeds through one or more cycles, each cycle resulting in polynucleotide replication. A linear amplification reaction cycle typically comprises the steps of denaturing the double-stranded template, annealing the single primer or primers to the denatured template, and synthesizing polynucleotides from the primers. Thus the term “linear amplification reaction” as used herein is meant to include all of these steps. In the case of a single-primer linear amplification reaction, only one primer is used in each single-primer linear amplification reaction. Linear amplification reactions used in the methods of the invention differ significantly from the polymerase chain reaction (PCR). The polymerase chain reaction produces an amplification product that grows exponentially in amount with respect to the number of cycles. Linear cyclic amplification reactions differ from PCR in that the amount of amplification product produced in a linear cyclic amplification reaction is linear with respect to the number of cycles performed. The reaction product accumulation rate laws differ because the products of each cycle in a PCR reaction are templates for the next cycle, while only the parentals are templates in linear amplification. As in PCR, a linear amplification reaction cycle typically comprises the steps of denaturing the double-stranded template, annealing the single primer or primers to the denatured template, and synthesizing polynucleotides from the primers. The cycle may be repeated several times so as to produce the desired amount of newly synthesized polynucleotide product. Although linear amplification reactions differ significantly from PCR, guidance in performing the various steps of linear cyclic amplification reactions can be obtained from reviewing literature describing PCR and other polymerase based methods including, PCR: A Practical Approach, M. J. McPherson, et al., IRL Press (1991), PCR Protocols: A Guide to Methods and Applications, by Innis, et al., Academic Press (1990), and PCR Technology: Principals and Applications of DNA Amplification, H. A. Erlich, Stockton Press (1989). PCR is also described in many U.S. Patents, including U.S. Pat. Nos. 4,683,195, 4,683,202; 4,800,159; 4,965,188; 4,889,818; 5,075,216; 5,079,352; 5,104,792, 5,023,171; 5,091,310; and 5,066,584, which are hereby incorporated by references. Many variations of amplification techniques are known to the person of skill in the art of molecular biology. These variations include rapid amplification of DNA ends (RACE-PCR), amplification refectory mutation system (ARMS), PLCR (a combination of polymerase chain reaction and ligase chain reaction), ligase chain reaction (LCR), self-sustained sequence replication (S SR), Q-beta amplification, and strand displacement amplification (SDA), and the like. A person of ordinary skill in the art may use these methods to modify the linear amplification reactions used in the methods of the invention.

The term “mutagenic primer” refers to an oligonucleotide primer used in a linear amplification reaction and single-primer linear amplification reaction, wherein the primer does not precisely match the target hybridization sequence. The mismatched nucleotides in the mutagenic primer are referred to as mutation sites with respect to the mutagenic primer. Thus, during the amplification reaction, the mismatched nucleotides of the primer are incorporated into the amplification product thereby resulting in the synthesis of a mutagenized DNA strand comprising the mutagenic primer that was used to prime synthesis mutagenizing the target sequence. The term “oligonucleotide” as used herein with respect to mutagenic primers is used broadly. Oligonucleotides include not only DNA but various analogs thereof. Such analogs may be base analogs and/or backbone analogs, e.g., phosphorothioates, phosphonates, and the like. Techniques for the synthesis of oligonucleotides, e.g., through phosphoramidite chemistry, are well known to the person ordinary skilled in the art and are described, among other places, in Oligonucleotides and Analogues: A Practical Approach, ed. Eckstein, IRL Press, Oxford (1992). Preferably, the oligonucleotide used in the methods of the invention are DNA molecules.

In accordance with the invention, parental DNA strands used as templates during linear amplification reactions may optionally be digested during the process of the invention. By performing the digestion step, the number of transformants containing non-mutagenized oligonucleotides is reduced. The term “digestion” as used herein in reference to the enzymatic activity of a selection enzyme is used broadly to refer both to (i) enzymes that catalyze the conversion of a polynucleotide into polynucleotide precursor molecules and to (ii) enzymes capable of catalyzing the hydrolysis of at least one bond on polynucleotides so as to interfere adversely with the ability of a polynucleotide to replicate (autonomously or otherwise) or to interfere adversely with the ability of a polynucleotide to be transformed into a host cell. Restriction endonucleases are an example of an enzyme that can “digest” a polynucleotide. Typically, a restriction endonuclease that functions as a selection enzyme in a given situation will introduce a specific single cleavage into the phosphodiester backbone of the template strands that are digested.

The term “selection enzyme” refers to an enzyme capable of catalyzing the digestion of a parental DNA used as the template in the linear amplification reactions, but not significantly digesting newly synthesized DNA strands. Examples of selection enzymes include restriction endonucleases. A preferred selection enzyme for use in the parental strand digestion step is the restriction endonuclease Dpn I, which cleaves the polynucleotide sequence GATC only when the adenine is methylated (6-methyl adenine). Other restriction endonucleases suitable for use in the parental strand digestion step include Nan II, NmuD I, and NmuE I. However, restriction endonucleases for use as selection enzymes in the digestion step do not need to be isoschizomers of Dpn I. Suitable selection enzymes are provided with commercially available mutagenesis kits such as the QuikChange® Site Directed Mutageneisis System kit supplied by Stratagene (La Jolla, Calif.). A variety of suitable selection enzymes may be purchased from a myriad of companies as is known in the art.

The term “double-stranded mutagenized DNA intermediate” as used herein refers to double-stranded DNA structures formed by annealing the first mutagenized DNA strand formed in the subject methods to the second mutagenized DNA strand When a double-stranded mutagenized DNA intermediate is transformed into a host cell, host cell enzymes are able to repair nicks in the molecule so as to provide a closed circular double-stranded DNA that corresponds to the original circular DNA molecule for mutagenesis that has been modified to contain the specific site-directed mutation or mutations of interest.

In one embodiment of the invention, the invention may be carried out using the reagents provided in commercially available mutagenesis kits such as Stratagene's QuikChange® II XL Site Directed Mutagenesis Kit. However, the present invention may be practiced without the use of a commercially available kit so long as a high fidelity polymerase and high competence cells are used in the present method. The reagents suitable for use in as described herein may also be purchased separately from a number of companies as is known in the art.

The methods of the invention employ pairs of mutagenic primers consisting of a first mutagenic primer and a second mutagenic primer. The mutagenic primers are about 20-50 bases in length, more preferably about 25 to 45 bases in length. However, in certain embodiments of the invention, it may be necessary to use mutagenic primers that are less than 20 bases or greater than 50 bases in length so as to obtain the mutagenesis result desired. The first and second mutagenic primers may be of the same or different lengths; however, in a preferred embodiment, the first and second mutagenic primers are the same length. The mutagenic primers are preferably not phosphorylated, however in some embodiments, 5′phosphorylation may be desirable. 5′ phosphorylation may be achieved by a number of methods well known to a person of ordinary skill in the art, e.g., T-4 polynucleotide kinase treatment.

The first and second mutagenic primers contain one or more mutagenic sites, i.e., mismatch locations with respect to the target DNA sequence to be mutagenized. The mutagenic site (or sites) may be used to introduce a variety of mutation types into the DNA sequence for mutagenesis. Such mutations include substitutions, insertions, and deletions. The principle of site-directed mutagenesis with single oligonucleotide primers is well known to the person of ordinary skill in the art, and can be found, for example, in Sambrook et al., Molecular Cloning: A Laboratory Manual, Second Edition, Cold Spring, Cold Spring Harbor, N.Y. (1989) and Wu et al., Recombinant DNA Methodology, Academic Press, San Diego, Calif. (1989). This information may be used to design the mutagenic sites in the first and second mutagenic primers employed in the subject methods.

The first and second mutagenic primers of the invention are either completely complementary to each other or partially complementary to each other. Preferably, the first and second mutagenic primers are selected so as to be completely complementary to each other. When the first and second mutagenic primers are partially complementary to each other, the region of complementarity should be contiguous. In embodiments of the invention in which the first and second mutagenic primer are partially complementary to one another, the region of complementarity must be sufficiently large to permit the mutagenic primers to anneal to the DNA molecule for mutagenesis; preferably, although not necessarily, the region of complementarity is at least 50% of the length of the primer (50% of the larger primer when the first and second primer are of different lengths). The mutagenic sites of the first and second mutagenic primers are located in or near the middle of the primer. Preferably, the mutagenic sites are flanked by about 10-15 bases of correct, i.e., non-mismatched, sequence so as to provide for the annealing of the primer to the template DNA strands for mutagenesis. In preferred embodiments of subject methods, the GC content of mutagenic primers is at least 40%, so as to increase the stability of the annealed primers. Preferably, the first and second mutagenic primers are selected so as to terminate in one or more G or C bases. Very high GC content (over 70%), or runs of more than five successive GC bases, are not desirable since this decreases specificity.

Synthesis of the first and second mutagenized DNA strands takes place during the synthesis phase of the single-primer linear amplification reaction(s). However, prior to the synthesis phase of the reaction, the single-primer linear amplification reaction necessarily includes the steps of denaturing the double-stranded DNA followed by annealing of the primer that is present in the reaction as described earlier.

The linear cyclic amplification reaction may be catalyzed by a thermostable or non-thermostable high-fidelity polymerase. Polymerases for use in the linear cyclic amplification reactions of the subject methods have the property of not displacing the mutagenic primers that are annealed to the template. Preferably, the polymerase used is a thermostable polymerase. The polymerase used may be isolated from naturally occurring cells or may be produced by recombinant DNA technology. The use of Pfu DNA polymerase (Stratagene, La Jolla, Calif.), a DNA polymerase naturally produced by the thermophilic archae Pyrococcus furiosus, is particularly preferred for use in the linear cyclic amplification reaction steps of the claimed invention. Pfu DNA polymerase is exceptionally effective in producing first and second mutagenized DNA strands of the appropriate length for formation of the desired double-stranded mutagenized DNA intermediates. Other high fidelity polymerases suitable for use in the present invention include but are not limited to, KOD HiFi (EMD Biosciences Inc., Madison, Wis.), Phusion™ High Fidelity Polymerase (Finnzymes, Espoo, Finland).

Single-primer linear amplification reactions as employed in the methods of the invention are preferably carried out to the limits imposed by the polymerase properties and the amplification conditions. Preferably the number of cycles in the linear cyclic amplification reaction step is at least 10 cycles, more preferably at least 20 cycles, and can be 25 or more cycles. The limitation on optimum cycle number is specific to the application and is imposed by runaway PCR artifact triggered by low probability nonspecific binding. This is a problem primarily in very CG rich regions, and can be overcome by decreasing the number of cycles and increasing the template concentration.

Appropriate reaction conditions are maintained throughout the various stages of the invention to maximize desirable reaction products produced at each stage while minimizing the production of artifact. The process of forming double-stranded mutagenized DNA intermediates should proceed for a period of time sufficient to produce a convenient number of double-stranded mutagenized DNA intermediates to provide a convenient number of clones in the subsequent transformation steps. Generally, incubation for one to two hours at 37° C. will be sufficient in most embodiments of the invention. However, these time and temperature parameters may be readily varied by the person of ordinary skill in the art so as to take into account factors such as DNA concentration, the GC content of the DNA molecules, etc.

After the double-stranded mutagenized DNA intermediate formation step is completed, the reaction mixture, or a portion thereof, may be used to transform competent or preferably ultracompetent single-cell microorganism host cells. It is not necessary to perform a ligation reaction prior to transformation of the host cells. The absence of a ligation step requirement serves to reduce the time and expense required to carry out the methods of the invention as compared with conventional methods of site directed mutagenesis. The host cells may be prokaryotic or eukaryotic. Preferably the host cells are prokaryotic, more preferably, the host cells for transformation are E. coli cells. Techniques for preparing and transforming competent single cell microorganisms are well know to the person of ordinary skill in the art and can be found, for example, in Sambrook et al., Molecular Cloning: A Laboratory Manual Coldspring Harbor Press, Coldspring Harbor, N.Y. (1989), Harwood Protocols For Gene Analysis, Methods In Molecular Biology Vol. 31, Humana Press, Totowa, N.J. (1994), and the like. Frozen competent cells may be transformed so as to make the methods of the invention particularly convenient.

Another aspect of the invention is to provide kits for performing the combinatorial mutagenesis methods of the invention. The kits of the invention provide one or more of the enzymes or other reagents for use in performing the subject methods. The kits may contain reagents in pre-measured amounts so as to ensure both precision and accuracy when performing the subject methods. Kits may also contain instructions for performing the methods of the invention. In one embodiment, kits of the invention comprise at least one polymerase and instructions for carrying out the method. The kits may also comprise a DNA vector comprising a cloning site, ultracompetent cells and blocking oligonucleotides complementary to regions of the DNA vector. Kits of the invention may also comprise individual nucleotide triphosphates, mixtures of nucleoside triphosphates (including equimolar mixtures of dATP, dTTP, dCTP and dGTP), and concentrated reaction buffers. In a preferred embodiment, the kits comprise at least one DNA polymerase, concentrated reaction buffer, a nucleoside triphosphate mix of the four primary nucleoside triphosphates in equal amounts, frozen competent or ultracompetent cells and instructions for carrying out the method.

One skilled in the art will appreciate the many advantages that the method of the invention provides. For example, the improved site-directed mutagenesis methods of the invention are useful in protein and enzyme engineering technologies for the production of industrial proteins and enzymes such as detergent enzymes, enzymes useful for neutralizing contaminants and enzymes useful as fuel additives. Likewise, the methods of the invention are useful in protein engineering technologies for the production of proteins and enzymes useful and the food and life sciences industries such as primary and secondary metabolites useful in the production of antibiotics, proteins and enzymes for the food industry (bread, beer) and combinatorial arrays of proteins for useful in generating multiple epitopes for vaccine production.

The methods of the invention can also be used in the production of mutagenized fusion proteins. A DNA sequence targeted for mutagenesis is tagged or fused with the DNA sequence encoding a known protein (e.g. maltose binding protein (MBP) or green fluorescent protein (GFP)). For example, vectors with a GFP gene adjacent to a cloning site would allow easy conversion of a vector for expression of a target gene to one of several possible target gene-GFP mutants with different linkers. These in turn could be targeted to different cell locations by modifications of the opposite (usually N) terminus. Kits designed identify fusion proteins are advantageously used to identify and isolate the mutagenized protein of interest in vitro (western blots) or in tissue using anti-GFP.

The following examples are offered by way of illustrating the invention and not by way of limitation.

### EXAMPLES

Materials and Methods:

In one set of experiments, pCWori+ containing genes for endothelial and neuronal nitric oxide synthase (eNOS and nNOS) were used as the templates for mutagenesis. These systems are ˜10 kB in length; mutagenesis by conventional methods is extremely tedious. and the system represents a significant challenge. A second set of experiments introduced mutations into much smaller small heat shock protein superfamily genes in pACYC184T7, a system of 4.8 kB. Primers for the mutations were synthesized and purified by polyacrylamide gel electrophoresis (PAGE) by One Trick Pony/Ransom Hill Biosciences (www.ransomhill.com).

A single-stage of single-primer linear amplification reaction was carried out separately for the forward and reverse primers as described in the specification above. 10-25 cycles of linear amplification were carried out to produce a reasonable number of copies, which are mixed and cooled to anneal the strands. The initial denaturation was carried out at about 94° C. for 2 minutes, subsequent denaturations were for 50 s at 94° C. Annealing was 50 s at 60° C.; extension was at 68° C. for 1-2 min/Kb template length. After mixing, the reaction product was denatured at 94° C. for 1 min., annealed for 7 min at 60° C., and cooled to 37° C. for 1-2 hr. The Stratagene Dpn1 digestion step can be inserted at any time durine the 37° C. incubation.

In the experiments using the pCWori+ system, transformation of Stratagene Gold ultracompetent cells by 10 μl of the product produced colonies with appropriate antibiotic resistance when plated. Sequencing of the plasmid DNA was done to confirm the production of mutants. In the experiments with the smaller pACYC184T7 system. Stratagene blue cells with a lower transformation efficiency were used.

Results

Sequences of target regions within parental NOS genes are shown in table I, along with forward and reverse primers for each trial. Mutant sequences are indicated in bold type. Primer design criteria in general followed Stratagene's recommendations for the QCM procedure (Tm˜78 C). (It is not necessary to adhere strictly to this recommendation in carrying out the present invention; lower temperatures are advantageous in AT rich regions.) After 15-25 rounds of linear amplification using one primer each in separate tubes. products were mixed and annealed without additional polymerase steps. 45 μl of ultra gold competent cells were transformed with 2-4 μl of the product; cells were plated on ampicillin containing agar, typically yielding 50-100 colonies per plate. Two or three colonies were selected at random for each mutant, and cultures were grown overnight in 1-3 ml of terrific broth. DNA was obtained by a plasmid miniprep, and the presence of mutations was evaluated by sequencing.

The results are summarized in Table I. For all mutagenesis trials, including insertion and deletion mutants as well as point mutations, a 96% mutation rate was obtained. 8 colonies of transformants carrying plasmids produced after 25 rounds of amplification were picked; none of these colonies contained parental plasmids. The high success rate cannot be attributed solely to dam methylation selection; as Wang and Malcolm (supra) pointed out double stranded hybrids containing a single parental strand are also protected. The success rate in case is likely attributed to swamping of parentals by large numbers of mutant strands produced in high efficiency linear amplification. Absent effective competing reactions complete copying of parental templates during each cycle should produce 96.15% mutants after 25 cycles. The Obtaining mutational frequencies approaching theoretical values in these trials was a surprising and desirable result. In most cases it is unnecessary to further suppress parentals. In comparison, previous attempts to produce point mutants in pCWori+ eNOS using the QCM procedure resulted only a ˜20% success rate.

The results of a second set of mutagenesis experiments using the pACYC184T7 vector with αA and αB crystallin and HSP16.5 genes are summarized in table II. In all, a series of ten mutations consisting of four to ten base insertions and deletions was successfully carried out. The overall frequency of parentals was higher in this set of experiments than in the pCWori+/NOS experiments; overall, 70% of the selected colonies carried the mutations. The frequency distribution of parentals was well described by random sampling with replacement with the assumption that 30% of the transformants were parentals.

The difference between the two experiments can be accounted for by the cell line used in the transformations. The Gold Ultracompetent cells used with the pCWori+ experiments were recommended for large vector/gene combinations, and were efficiently transformed by the linearized mutant bearing plasmids. The Blue Ultracompetent cells recommended as sufficient for smaller systems were in fact effective enough to allow production of all ten mutants with selection of only a few colonies, but the results suggest a bias of five of six fold towards transformation with circular parental gene bearing plasmids in comparison to linearized mutant bearing plasmids. Typically about ⅓ as many colonies were obtained per plate with the Blue cells.

### Tables

TABLE I

Primers and results from representative pCWori+/NOS mutagenesis
5 experiments. Results column represents (mutants confirmed)/(clones selected). In
both tables, insertions are represented by boldface type, deletions by boldface type
in parentheses, and substituted codons are in italics and underlined.

| | | |
|---|---|---|
| | | Sequencing |
| Mutant | Primer Sequence | Results |

| | | | |
|---|---|---|---|
| nNOS₍₋₁₀₇₁₋₁₀₇₃₎ | F: CTG GAG GAG AGG AAC (ACT GCT CTG) GGT GTC ATC AGT AAT | (SEQ ID 1) | 2/2 |
| | R: ATT ACT GAT GAC ACC (CAG AGC AGT) GTT CCT CTC CTC CAG | (SEQ ID 2) | |
| nNOS_{(G1047Y)} | F: GAG AGG AAC ACT GCT CTG TATGTC ATC AGT | (SEQ ID 3) | 3/4* |
| | R: CA ATT ACT GAT GAC ATACAG AGC AGT GTT C | (SEQ ID 4) | |
| nNOS_{(G1074D)} | F: GAG AGG AAC ACT GCT CTG GATGTC ATC AGT | (SEQ ID 5) | 3/4* |
| | R: CA ATT ACT GAT GAC ATCCAG AGC AGT GTT C | (SEQ ID 6) | |
| eNOS₍₋₈₄₀₎ | F: GC AGC CCA GGC (GGC) CCT CCT CCC AGC TGG G | (SEQ ID 7) | 1/1 |
| | R: GC TGG GAG GAG G (GCC) GC CTG GGC TGC CTT TCT C | (SEQ ID 8) | |
| eNOS_{(G840Y),} | F: C AGC CCA GGC TACCCT CCT CCC AGC TGG | (SEQ ID 9) | 2/2 |
| | R: C TGG GAG GAG GGTAGC CTG GGC TGC CTT TC | (SEQ ID 10) | |
| eNOS_{(G840D),} | F: C AGC CCA GGC GACCCT CCT CCC AGC TGG | (SEQ ID 11) | 3/3 |
| | R: C TGG GAG GAG GGTCGC CTG GGC TGC CTT TC | (SEQ ID 12) | |
| nNOS_{(G1072-1077),} | F: GAG GAG AGG AAC ACT (GCT CTG GGT GTC ATC AGT) | (SEQ ID 13) | 4/4 |
| | AAT TGG AAG GAT GAA: | | |
| | R: TTC ATC CTT CCA ATT (ACT GAT GAC ACC CAG AGC) | (SEQ ID 14) | |
| | AGT GTT CCT CTC CTC: | | |

*These two clones were constructed using only 10 cycles of linear amplification

TABLE 11

Primers and results from representative pACYC184T7/sHSPmutagenesis
experiments. Results column represents (mutants confirmed) /(clones selected). In
both tables, insertions are represented by boldface type, deletions by boldface type
in parentheses, and substituted codons are in italics and underlined.

| | | |
|---|---|---|
| | | Sequencing |
| Clone Name | Primer Sequence | Results |

| | | | |
|---|---|---|---|
| Alpha A Crystallin- | F: GCG CCC TCG TCC TAA TCT CGA GCA CCA CC-3′ | (SEQ ID 15) | 3/5 |
| | R: GGT GGT GCT CGA GAT TAG GAC GAG GGC GC | (SEQ ID 16) | |
| Alpha B Crystallin- | F: GCC CCC AAG AAG TAA TCT CGA GCA CCA CCA CC | (SEQ ID 17) | 3/3 |
| | R: GGT GGT GGT GCT CGA GAT TAT TTC TTG GGG GC | (SEQ ID 18) | |
| MjHSP16.5- | F: GGA ATC AAC ATT GAA TAA TCT CGA GCA CCA CCA CC | (SEQ ID 19) | 2/3 |
| | R: GGT GGT GGT GCT CGA GAT TAT TCA ATG TTG ATT CC | (SEQ ID 20) | |
| Alpha B Crystallin-KK- | F: CAC CGC AGC CCC C (AAG AAG TAA T) CT CGA GCA CCA CCA C: | (SEQ ID 21) | 3/4 |
| deletion | | | |
| | R: GTG GTG GTG CTC GAG (AT TAC TTC TT) GGG GGC TGC GGT G: | (SEQ ID 22) | |

The patent and scientific literature referred to herein establishes the knowledge that is available to those with skill in the art. All United States patents and published or unpublished United States patent applications cited herein are hereby incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of introducing mutations into a DNA molecule, comprising the steps of:
(a) adding a first mutagenic primer to a first parental DNA molecule in a first reaction;
(b) adding a second mutagenic primer to a second parental DNA molecule in a second reaction, wherein the first and second parental DNA molecules are identical, and wherein the first mutagenic primer comprises a region that is complementary to the second mutagenic primer and the first and second mutagenic primers each contain at least one mutation site with respect to the first and second parental DNA molecules, wherein the mutation site is located within the region that is complementary between said first and second mutagenic primers;
(c) synthesizing by means of at least one cycle of a single-primer linear amplification reaction a first mutagenized DNA strand comprising the first mutagenic primer in the first reaction;
(d) synthesizing by means of at least one cycle of a single-primer linear amplification reaction a second mutagenized DNA strand comprising the second mutagenic primer in the second reaction;
(e) combining the reaction products from (c) with the reaction products from (d); and
(f) annealing the first mutagenized strand with the second mutagenized strand to form a double-stranded mutagenized DNA intermediate.

2. The method of claim 1, further comprising the step of transforming a host cell with the double-stranded mutagenized DNA intermediate.

3. The method according to claim 1 wherein the single-primer linear amplification reactions of steps (c) and (d) are each repeated for at least 25 cycles.

4. The method according to claim 1, further comprising a digestion step before or during the annealing step wherein the first and second parental DNA molecules are digested with a selection enzyme.

5. The method of claim 4 wherein the selection enzyme is DpnI.

6. The method of claim 4 wherein the selection enzyme is a restriction endonuclease or a glycosylase.

7. The method of claim 1 wherein the single-primer linear amplification reactions of steps (c) and (d) are catalyzed by Pfu DNA polymerase.

8. The method of claim 1 further comprising reacting the double-stranded mutagenized DNA intermediate of step (f) with a ligase.

9. A method of introducing mutations into a DNA molecule, wherein the DNA molecule is double-stranded and circular, comprising the steps of:
(a) adding a first mutagenic primer to a first DNA molecule in a first reaction;
(b) adding a second mutagenic primer to a second DNA molecule in a second reaction, wherein the first and second DNA molecules are identical, and wherein the first mutagenic primer comprises a region that is complementary to the second mutagenic primer and the first and second mutagenic primers each contain at least one mutation site with respect to the DNA molecule, wherein the mutation site is located within the region that is complementary between said first and second mutagenic primers;
(c) synthesizing by means of at least one cycle of a single-primer linear amplification reaction a first mutagenized DNA strand comprising the first mutagenic primer in the first reaction;
(d) synthesizing by means of at least one cycle of a single-primer linear amplification reaction a second mutagenized DNA strand comprising the second mutagenic primer in the second reaction;
(e) combining the reaction products from (c) with the reaction products from (d); and
(f) annealing the first mutagenized strand with the second mutagenized strand to form a double stranded mutagenized DNA intermediate.

10. The method of claim 9 further comprising the step of transforming a host cell with the double-stranded mutagenized DNA intermediate.

11. The method according to claim 9 wherein the single-primer linear amplification reactions of steps (c) and (d) are each repeated for at least 25 cycles.

12. The method according to claim 9 further comprising further comprising a digestion step before or during the annealing step wherein the first and second parental DNA molecules are digested with a selection enzyme.

13. The method according to claim 12 wherein the selection enzyme is DpnI.

14. The method of claim 12 wherein the selection enzyme is a restriction endonuclease or a glycosylase.

15. The method of claim 9 wherein the linear amplification reactions of steps (c) and (d) are catalyzed by Pfu DNA polymerase.

16. The method of claim 9 wherein said first and second mutagenic primers are completely complementary to each other.

17. A kit for use in the method of claim 1 comprising a DNA polymerase, and instructions for carrying out the method.

18. The kit of claim 17 further comprising competent or ultracompetent cells.

19. The kit of claim 17 further comprising identical DNA vectors comprising identical cloning sites.

20. The kit of claim 17 further comprising individual nucleotide triphosphates or mixtures of nucleoside triphosphates.

21. A method of using a kit comprising a DNA polymerase and instructions for carrying out the method in a method comprising the steps of:
(a) adding a first mutagenic primer to a first parental DNA molecule in a first reaction;
(b) adding a second mutagenic primer to a second parental DNA molecule in a second reaction, wherein the first and second parental DNA molecules are identical, and wherein the first mutagenic primer comprises a region that is complementary to the second mutagenic primer and the first and second mutagenic primers each contain at least one mutation site with respect to the first and second parental DNA molecules, wherein the mutation site is located within the region that is complementary between said first and second mutagenic primers;
(c) synthesizing by means of at least one cycle of a single-primer linear amplification reaction a first mutagenized DNA strand comprising the first mutagenic primer in the first reaction;
(d) synthesizing by means of at least one cycle of a single-primer linear a mplification reaction a second mutagenized DNA strand comprising the second mutagenic primer in the second reaction;
(e) combining the reaction products from (c) with the reaction products from (d); and
(f) annealing the first mutagenized strand with the second mutagenized strand to form a double-stranded mutagenized DNA intermediate.
